(19) United States
(12) Patent Application Publication
(10) Pub. No.: US 2018/0221451 A1
(43) Pub. Date: Aug. 9, 2018

(54) LONG ACTING LIRAGLUTIDE COMPOSITIONS

(71) Applicant: Sun Pharma Advanced Research Company Limited, Mumbai (IN)

(72) Inventors: Ajay Jaysingh Khopade, Baroda (IN); Arindam Halder, Baroda (IN); Vivek Patel, Baroda (IN)

(73) Assignee: Sun Pharma Advanced Research Company Limited, Mumbai (IN)

(21) Appl. No.: 15/750,705

(22) PCT Filed: Aug. 12, 2016
(86) PCT No.: PCT/IN2016/050272

§ 371 Date: Feb. 6, 2018

(30) Foreign Application Priority Data
Aug. 13, 2015 (IN) 3069/MUM/2015

(51) Int. Cl. A61K 38/26; A61K 9/16; A61P 3/10

(52) U.S. Cl. CPC A61K 38/26; A61K 9/1647; A61P 3/10; A61K 9/1641; A61K 9/1658

(57) ABSTRACT

The present invention relates to a composition comprising particles, wherein said particles comprise Poly(lactide-co-glycolide) polymer, therapeutically effective amount of liraglutide or a pharmaceutically acceptable salt thereof and a hydrophilic particle size modulating agent, wherein, the composition is free of added divalent metal ions. The composition provides effective blood glucose control for about 7 days when administered as once a week formulation to a period of about 30 days after single administration of a monthly dose to a patient in need thereof.

### FIELD OF THE INVENTION

The invention relates to long acting liraglutide compositions, methods of making them and use of such composition in the treatment of metabolic diseases.

### BACKGROUND OF THE INVENTION

Diabetic mellitus is a disease of metabolic dysregulation, most notably abnormal glucose metabolism, accompanied by characteristic long term complications. It's a chronic disease requiring long term medications. Different parenteral anti-diabetic medications are available in market including human insulin and different GLP-1 agonists.

The natural GLP-1 is a gut hormone with therapeutic potential in the treatment of Type 1 and Type 2 diabetes and the treatment of obesity. The natural GLP-1 has a short half-life of only few minutes in the body as it is rapidly degraded by dipeptidyl peptidase-4 enzyme.

Many GLP-1 agonists were developed by modifications to natural GLP-1 to overcome the problem of its short half-life. One of the approaches used was substitution of one or more amino acids of the GLP-1 polypeptide and attachment of a lipophilic substituent to these peptides. These lipophilic substituted GLP-1 agonists showed protracted action when injected.

U.S. Pat. No. 6,268,343 disclosed such fatty acid acylated GLP-1 agonists. One particular example includes liraglutide. Liraglutide is a once daily human GLP-1 analog (97% homology). Liraglutide is (Arg34, Lys²⁶ (N^{ε}-(γ-GLu(N-hexadecanoyl)))-GLP-1(7-37). In United States, Liraglutide is approved as a once daily subcutaneous injection to improve glycemic control in adults with type 2 diabetes mellitus. It is also approved in United States for weight management in adult patients.

Exenatide, a 39 amino acid peptide, is a modified GLP-1 agonist which is available as a twice daily injection for the treatment of Type 2 diabetes mellitus. An extended release formulation of exenatide for once-weekly administration was approved in United States in 2012. This once-weekly formulation comprises of exenatide and sucrose encapsulated in microspheres.

US20140220134 disclosed once-monthly formulation of exenatide using extended release microspheres suspension comprising poly(lactide-co-glycolide) polymer in medium chain triglycerides.

However, in spite of the available therapies, there is s still a need to lower the frequency of injections for the patients and to provide a long acting composition of liraglutide for once weekly or biweekly or monthly administration.

### SUMMARY OF THE INVENTION

The present inventors have found advantageous long acting liraglutide compositions.

Particularly, the present invention provides a composition comprising particles, wherein the said particles comprises,

a) Poly(lactide-co-glycolide) polymer

b) therapeutically effective amount of liraglutide or a pharmaceutically acceptable salt thereof; and

c) a hydrophilic particle size modulating agent

wherein, the composition is free of added divalent metal ions.

In another embodiment, the present invention provides a method of reducing glucose levels in a patient in need thereof, comprising administering the composition of the present invention.

In yet another embodiment, the invention provides a long acting composition of liraglutide for once weekly or biweekly or monthly administration. In a further embodiment, the invention provides methods of preparing such compositions.

### DESCRIPTION OF THE FIGURES

FIG. 1 depicts Scanning Electron Microspcopy (SEM) images of Liraglutide particles of Example 1.

FIGS. 2(a), (b), (c) and (d) depicts Nikon microscopic images of Liraglutide particles of Comparative Example 1, Example 1, Example 2 and Example 3 of the invention respectively.

FIGS. 3(a) and (b) depicts images of coacervates in the vessel in Comparative Example 1 and Example 1 respectively.

FIG. 4 depicts the mean blood glucose reduction in db/db mice (20 mg/kg) after single subcutaneous injection of liraglutide particles of Example 1, 4 and 5 vs placebo.

FIG. 5 depicts the reduction in HbA1C in db/db mice on administration of liraglutide particles of Examples 1

FIG. 6 depicts the reduction in HbA1C in db/db mice on administration of liraglutide particles of Examples 4 and 5.

### DETAILED DESCRIPTION OF THE INVENTION

The invention provides a long acting composition of liraglutide and method of making and using the same. The composition of the invention can be used in the treatment of metabolic diseases such as diabetes and obesity.

The present inventors have advantageously discovered a long acting composition of liraglutide. The composition offers advantage over Victoza® in that it may be administered at a lower frequency, thus providing convenience to patients and thereby increasing patient compliance, further providing an effective blood glucose control over a longer period of time. The composition of the invention provides a long acting composition of liraglutide for once weekly or biweekly or monthly administration.

In one embodiment, the present invention provides a composition comprising particles, wherein the said particles comprises,
a) Poly(lactide-co-glycolide) polymer
b) therapeutically effective amount of liraglutide or a pharmaceutically acceptable salt thereof; and
c) a hydrophilic particle size modulating agent

wherein, the composition is free of added divalent metal ions.

The term “long acting” as used herein, refers to the duration of action of composition of the liraglutide as disclosed herein. More specifically, it refers to the period of time after administration of a dose of liraglutide composition of the present invention for which blood glucose levels are controlled. The composition of the present invention can be suitably formulated to provide effective blood glucose control over a period of about 7 days when administered as once a week formulation to a period of about 30 days after single administration of a monthly dose to a patient in need thereof. Preferably, the composition of the present invention may be suitably formulated to obtain the desired blood levels of liraglutide such that it provides effective blood glucose control over an intended dosage interval which may be for about a week to about a month after a single administration. In a more preferred embodiment, the composition of the present invention provides effective blood glucose control for about a month after single administration such that the composition may be administered as once a month injection.

The term “particles” as used herein includes microspheres, microparticles, and the like. The particle comprises the Poly(lactide-co-glycolide) polymer and the therapeutically acceptable amount of liraglutide or a pharmaceutically acceptable salt thereof and the hydrophilic size modulating agent. In one embodiment, the particle comprises a coating of Poly(lactide-co-glycolide) polymer surrounding the core comprising liraglutide or its pharmaceutically acceptable salt and the hydrophilic size modulating agent. The coating of PLGA may be a uniform or non-uniform, continuous or a discontinuous coating. In another embodiment, the core comprising liraglutide and the hydrophilic size modulating agent may be dispersed in the PLGA matrix. PLGA acts as a rate controlling agent and is responsible for providing sustained release of liraglutide oever an extended period of time. The hydrophilic size modulating agent also contributes to the sustained release property of the composition. The particles of the invention are preferably spherical in shape. The mean size range of the particle, measured in terms if diameter, is in the range from about 5 μm to about 250 μm. In a preferable embodiment the mean size of the particle is between 5-100 μm. The mean size of the particle is controlled by means of a hydrophilic size modulating agent. The particles may be suitably formulated to prepare a composition for administration to a patient in need thereof.

The term “added divalent metal ions” herein refers to divalent metal ions that may be added to the composition and the term does not include any divalent metal ion that may be available from any excipient or vehicle or as an impurity in the composition.

Liraglutide may be present in the composition in the form of base or in the form of its salts or mixtures thereof. Representative example of salts includes salts with suitable inorganic acids such as hydrochloric, hydrobromic, and the like. Representative examples of salts also includes salts with organic acids such as formic acid, acetic acid, propionic acid, lactic acid, tartaric acid, ascorbic acid and the like. Representative examples of salts also includes salt with base such as triethanolamine, diethylamine, meglumine, arginine, alanine, leucine, diethylethanolamine, olamine, triethylamine, tromethamine, choline, trimethylamine, taurine, benzamine, methylamine, dimethylamine, trimethylamine, methylethanolamine, propylamine, isopropylamine, adenine, guanine, cytosine, thymine, uracil, thymine, xanthine, hypoxanthine and like. In a preferred embodiment, liraglutide is present as liraglutide acetate. In another preferred embodiment, liraglutide is present as a tromethamine salt. Further the term “liraglutide” also include a mixture of liraglutide base with small amounts of acetic acid for eg. acetic acid may be present in less than 3% of weight of liraglutide and the present invention includes such form of liraglutide. Such forms of liraglutide are commercially available.

In another embodiment, liraglutide used in the composition may be in the form of a lyophilized mixture comprising liraglutide with parenterally acceptable amine base. The lyophilized mixture may be prepared by mixing liraglutide or a pharmaceutically acceptable salt thereof and a parenterally acceptable amine base in water for injection to form a solution and lyophilizing the solution to form the lyophilized mixture. The parenterally acceptable amine base may be selected from triethanolamine, diethylamine, meglumine, ornithine, lysine, arginine, alanine, leucine, diethylethanolamine, olamine, triethylamine, tromethamine, glucosamine, choline, trimethylamine, taurine, benzamine, trimethyl ammonium hydroxide, epolamine methylamine, dimethylamine, trimethylamine, methylethanolamine, propylamine, isopropylamine, and like. Preferably, the parenterally acceptable amine base is selected from tromethamine, histidine, lysine and arginine. The present inventors have observed that particles with a higher concentration of liraglutide can be prepared using the lyophilized mixture of liraglutide with a phrenterally acceptable amine base.

The amount of Liraglutide that may be present in the composition may be in an amount from about 5% w/w to about 40% w/w of the total weight of the particles used in the composition. In preferred embodiment the amount of liraglutide in a given composition may be present from about 5% w/w to about 15% w/w of the total weight of the particles used for making said composition. In another preferred embodiment, the amount of liraglutide may be between 16% w/w to about 25% w/w of the total weight of the particles used in making a composition. The amount is calculated in terms of equivalent of liraglutide base.

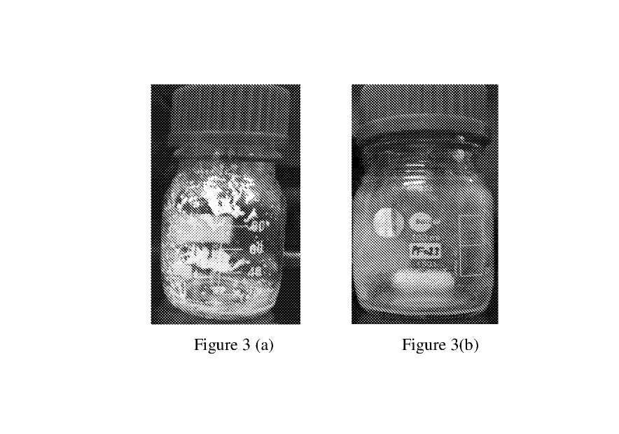

The term “hydrophilic particle size modulating agent” as used herein refers to agents which affects the size of the particle. The term “hydrophilic” herein refers to agents having affinity for water, which readily absorbs or dissolves in water and has a HLB value more than 8. The hydrophilic particle size modulating agent affects the size of the resulting particles such that the particle size is within the desired range. In a preferred embodiment, the particle size may be in the range of 50-100 μm. The present inventors have surprisingly found that the hydrophilic particle size modulating agent in the particles stabilizes and modulates the size of the particles. Further, during manufacturing of the particles this hydrophilic particle size modulating agents prevents coacervates from sticking to the wall of the vessel during the preparation of secondary emulsion (see FIG. 3). The hydrophilic particle size modulating agent also contributes to the protracted action of liraglutide. The hydrophilic particle size modulator may be present in an amount from about 0.01% to 5% w/w of the total weight of particles.

The hydrophilic particle size modulating agent may be selected from the group consisting of protamine, poloxamer, poly L-lysine, polyarginine, polyhistidine, polyethyleneimine, polypropyleneimine, polyvinyl alcohol, polyvinyl pyrrolidone, polyamidoamine, dextran-spermine, poly(L-lysine)-b-PEG copolymer, PolyVivo mPEG-PLGA diblock copolymers, PLGA-block-PEG-block-PLGA (poly(lactic acid-co-glycolic acid)-block-poly(ethylene glycol)-block-poly(lactic acid-co-glycolic acid) triblock copolymers or its pharmaceutically acceptable salts or mixtures thereof.

Preferably, the hydrophilic particle size modulating agent is selected from protamine, poloxamer, poly L-lysine, polyarginine, polyhistidine, or its pharmaceutically acceptable salts or mixtures thereof.

In a particularly preferred embodiment, protamine is used as the hydrophilic particle size modulating agent. It may be used as a base or salt such as protamine sulfate, protamine chloride or protamine acetate. In a preferred embodiment, protamine sulfate may be used. It may be present in an amount from about 0.01% w/w to about 1% w/w of the total weight of particles. In a preferred embodiment, it may be present in an amount from about 0.05% w/w to about 0.5% w/w of the total weight of particles.

In another embodiment, poloxamer is used as the hydrophilic particle size modulating agent. In a preferred embodiment, poloxamer 188 is used as the hydrophilic particle size modulating agent. In a preferred embodiment, poloxamer is present in an amount from about 0.1% w/w to about 5% w/w of the total weight of particles.

In yet another preferred embodiment, poly L-lysine is used as the hydrophilic particle size modulating agent. Poly L-lysine may be present in an amount from about 0.01% w/w to about 1% w/w of the total weight of particles. In a preferred embodiment, poly L-lysine is present in an amount from about 0.05% w/w to about 0.5% w/w of the total weight of particles.

The composition of the present invention comprises a Poly(lactide-co-glycolide) polymer (PLGA). Poly(lactide-co-glycolide) polymer in the composition of the invention is the rate controlling agent and is responsible for the “long acting” composition of the present invention. The hydrophilic size modulating agent also contributes to the rate controlling property of the long acting composition of the present invention. In a preferred embodiment, the PLGA has a lactide:glycolide ratio from 70:30 to 30:70, or from 60:40 to about 40:60 or about 50:50. In a more preferred embodiment, PLGA has a lactide:glycolide ratio of about 50:50. The molecular weight of PLGA used in the composition of the present invention may be in the range of about 10,000 Da to about 80,000 Da. Preferably, the molecular weight may be in the range from about 50,000 Da to about 70,000 Da. Further, PLGA may be used in the composition in an amount from about 50% to about 95% of the composition, preferably, it may be used in an amount from 70% to 90% of the composition. Usually, PLGA polymers may have D, L-lactide and glycolide monomer impurities. The inventors have found that the presence of these impurities may have a negative effect on the therapeutic efficacy of the composition. Thus, PLGA used in the composition of the present invention may be preferably, substantially free of residual D,L-lactide and glycolide monomers. More preferably, residual D, L-lactide and glycolide monomers may be less than 0.1%. Thus, PLGA may be purified to reduce the levels of residual D,L-lactide and glycolide monomers by PLGA purification methods. In one such method, PLGA is dissolved in acetone to form a solution to which water of injection is slowly added, to obtain a solid, which is filtered off. The resultant solid is then subjected to the same process using dichloromethane, instead of acetone, to give purified PLGA. Residual monomers may be evaluated by gas chromatography. This process yields PLGA with less than 0.1% of residual D,L-lactide and glycolide monomers. Alternatively, commercial sources of purified grade of PLGA with less than 0.1% of residual D,L-lactide and glycolide monomers may be used. It has been surprisingly found that the impurity profile of liraglutide is controlled to minimum by using purified PLGA wherein D.L-lactide and glycolide monomer are each less than 0.1% of PLGA. It was observed that with the use of PLGA with less than 0.1% residual D,L-lactide and glycolide monomers the total impurity levels of liraglutide were found to be less than 3% and further the known and unknown individual impurities were less than 0.5% as determined by HPLC.

In another embodiment of the present invention, a method for preparing particles of the composition of the invention is provided. The first step in method for preparing the particles comprises preparing an aqueous phase comprising liraglutide and a hydrophilic size modulating agent. Liraglutide may be present as a base, or its salt or it may added in the form of a lyophilized mixture with a parenterally acceptable amine base. Alternatively, the aqueous phase may be prepared by adding a lyophilized mixture comprising liraglutide, a parenterally acceptable amine base and hydrophilic size modulating agent, to water for injection. The lyophilized mixture of liraglutide with a parenterally acceptable amine base may be prepared by a process comprising the following steps:
a) preparing a solution of parenterally acceptable amine base in water for injection,
b) adding liraglutide or its acid addition salt to solution of step (a) while stirring to form a solution,
c) optionally sterilizing the aqueous solution by aseptic filtration, and
d) lyophilizing the solution to obtain a lyophilized mixture.

wherein the amount of parenterally acceptable amine base is such that the pH of the aqueous solution is in the range from about 6.7 to about 10. In the alternative method, the hydrophilic size modulating agent is added to the aqueous solution of step (b). The inventors have found that with the use of lyophilized mixture as stated above, particles with a higher concentration of liraglutide or its salt may be prepared. This is advantageous for the long acting compositions of the present invention as it may reduce the volume of the composition that may be need to be injected at a given time, especially for once a month composition. The concentration of liraglutide in the aqueous phase may be in the range from 2% w/w to about 25% w/w, preferably, the aqueous phase contains 5% w/w to 20% w/w of liraglutide.

In the second step of method of preparing the composition, an oil phase is prepared by dissolving PLGA in an oil phase. Preferably, the PLGA polymer can be present in the oil phase in a concentration ranging from about 2% w/w to about 20% w/w, preferably, from about 3% w/w to about 10% w/w of the oil phase. The solvent used in the oil phase is a solvent for PLGA. Such solvents are well known in the art. Preferably, solvent is selected from halogenated solvents such as dichloromethane, ethyl acetate, acetonitrile, methyl acetate, acetone, dimethylsulfoxide etc.

In third step, a primary water-in-oil emulsion is prepared by adding the aqueous phase of first step to an oil phase prepared in second step and sonicating or homogenizing the mixture of aqueous phase in oil phase. A suitable homogenizer may be used eg. Megatron homogenizer or kinematic homogenizer etc.

In Step four, a coacervation medium is added to the primary emulsion formed in the third step to form a secondary water-in-oil-in-oil emulsion. The coacervation medium is second oil phase which forms a distinct phase with the oil phase of PLGA. Suitable coacervation medium for use in the present invention includes, but are not limited to silicone oil, dimethicone, vegetable oil, mineral oil etc. The coacervation medium precipitates PLGA on the aqueous phase. Thus, PLGA forms a layer around the aqueous phase containing liraglutide and a hydrophilic size modulating agent, thereby forming particles with coating of PLGA surrounding the core comprising liraglutide and the hydrophilic size modulating agent. These particles are however soft and require hardening.

In step five, the microspheres prepared in step four above, is hardened by adding a quenching solvent to the microspheres of step four Suitable quenching solvent for use in the present invention includes, but is not limited to heptane, diethyl ether, hexane, cyclohexane, petroleum ether. In a preferable embodiment, the quenching solvent is a mixture of heptane and diethyl ether. Further, the solvents used in first oil phase and the coacervation medium were extracted using an extracting solvent. The extracting solvent is chosen such that is a non-solvent for the PLGA but is a solvent for the first oil phase and for the coacervating medium. Upon extraction using an extracting solvent, the particles precipitate out of the extracting solvent which may then be washed and dried. In a specific example, the method comprises:
a) Preparing an W/O emulsion comprising an aqueous phase of liraglutide, tromethamine and protamine sulfate in water for injection and an oil phase of Poly(lactide-co-glycolide) polymer in a halogenated solvent.
b) Further, combining the emulsion obtained in step (a) with the coacervation medium to form a W/O/O emulsion
c) Transferring the mixture to a quenching solvent or mixtures thereof,
d) Extracting the halogenated solvent with a extracting solvent, whereby the particles precipitates out, and
e) Drying, wetting and further drying the precipitated particles.

The particles formed above may be suitably formulated in a composition for administration to a person in need thereof. In one embodiment, the particles may be further formulated as an injection. An injection composition prepared according to the present invention comprises the particles as described herein with other excipients in an injection vehicle. In another embodiment, the particles can be suspended in the vehicle. The injection vehicle may be aqueous or non-aqueous vehicle suitable for parenteral administration. The excipient which may be used in the composition of the invention includes, but is not limited to viscosity enhancing agent, tonicity adjusting agent, buffers and/or wetting agent. The viscosity enhancing agent may be added in the composition to provide injectability of the composition through a needle ranging in diameter from 18-23 gauge. The viscosity enhancing agent may be selected from group comprising cellulose derivatives, such as hydroxypropyl cellulose, hydroxyethyl cellulose, hydroxypropyl methyl cellulose, methylcellulose, sodium carboxymethyl cellulose and its derivatives, polyvinylpyrrolidone (PVP), polysaccharides such as glycosaminoglycans, agar, pectin, alginic acid, dextran, starch and chitosan, proteins, poly(ethyleneoxide), polyhydroxy acids, polyanhydrides, polyamides, polycarbonates, polyalkylenes, polyalkylene glycols, polyalkylene oxides, polyvinyl alcohols, polyvinyl pyrrolidone and polyvinyl alcohol, polyvinyl ethers, polyvinyl esters, polyvinyl halides, polyvinylpyrrolidone, polysiloxanes, polyvinyl acetates, synthetic celluloses, polyacrylic acids, polybutyric acid, polyvaleric acid, and copolymers, derivatives, and the like; or mixtures thereof. Preferably, the viscosity enhancing agent is selected from carboxymethylcellulose sodium, polyvinylpyrrolidone (PVP) and hydroxypropylmethylcellulose (HPMC). The tonicity adjusting agent that may be used in the composition of the invention may be selected from sodium chloride, mannitol, propylene glycol etc. Preferably, the tonicity adjusting agent is sodium chloride. Suitable buffering agents for use in the composition herein include, but are not limited to, organic acid salts such as salts of citric acid, ascorbic acid, gluconic acid, carbonic acid, tartaric acid, succinic acid, acetic acid or phthalic acid; Tris, thomethamine hydrochloride, or phosphate buffer. Preferably, the buffer used in the composition may be sodium phosphate monobasic monohydrate and sodium phosphate dibasic heptahydrate. The composition may further contain a wetting agent. Suitable the wetting agent includes, polysorbate 20, polysorbate 40, polysorbate 80. In a preferred embodiment, the wetting agent is polysorbate 20.

The present invention also provides use of composition of the invention for use in the treatment of metabolic diseases. In a preferred embodiment, the composition of the invention is suitable for use in the treatment of diabetes. In another embodiment, the composition of the invention is suitable for use in the treatment of obesity. In another embodiment, the invention provides a method of reducing blood glucose level for a period of at least one month in a patient in need thereof. In another embodiment, the composition of the invention is suitable for use in reducing blood glucose levels in a patient in need thereof for a period of at least one week. The composition of the invention provides for maintenance of therapeutic levels of liraglutide after single administration over an extended period of time which may be for a week or two weeks or for a month. The composition is used for the treatment of diabetes by administration of composition once weekly, once biweekly or once monthly. In a preferred embodiment, the composition is used for the treatment of diabetes by administration of composition once monthly.

The composition of the invention may be administered by injection. Preferably, the composition of the invention may be administered by subcutaneous or intramuscular injection.

### EXAMPLES

The compositions of the present invention are illustrated as examples herein under. However, it is to be noted that the present disclosure is not limited to the illustrative examples but can be realized in various other ways.

### Comparative Example 1

| Sr. No. | Ingredients |
|---|---|
| 1 | Liraglutide |
| 2 | Tromethamine |
| 5 | Water for Injection |
| 5 | PLGA |
| 6 | Dichloromethane (MDC) |
| 7 | Dimethicone |
| 8 | n-Heptane |
| 9 | Di-ethyl ether |
| 10 | n-Heptane for-oil drying/solvent extraction |
| 11 | N₂ Drying at 2-8° C. for 0.5-1 h |
| 12 | 1% w/v Tween 80 solution in water |
| 13 | N₂ Drying at 25 ± 3° C. ° C. for 15-18 h |

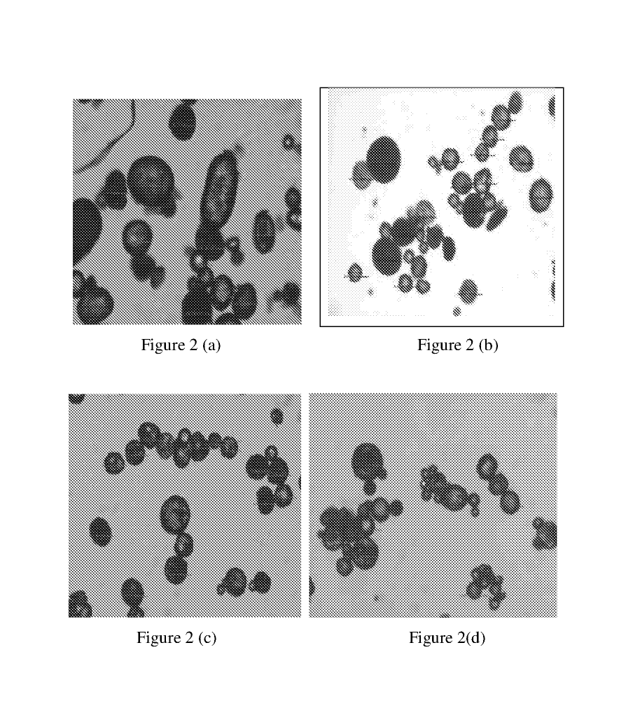

Liraglutide (100 mg), tromethamine (10 mg) were dissolved in water for injection (q.s. to 1 gm) to form the inner aqueous phase. The oil phase was prepared by dissolving PLGA (890 mg) in 13.94 g MDC. The aqueous phase was added to the oil phase using a homogenizer to form a primary emulsion. To this primary emulsion, 20.92 g Dimethicone was added immediately after primary emulsification under stirring to form the secondary emulsion (W/O/O). A solvent mixture was prepared by adding n-Heptane 10.95% w/v in di-ethyl Ether (ratio 1:9). To this mixture, the secondary emulsion was added under stirring. 40 g of n-Heptane was added to above mixture. n-Heptane and di-ethyl Ether mixture was then decanted. Fresh n-Heptane (100.5 g) was added at an ambient temperature over the sediment particles and stirred followed by decantation. This process was repeated twice. Finally, particles suspension in n-heptane was passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. Particles collected on the screen were dried and collected into a collection vessel. Elongated particles were observed under Nikon optical microscopy. Particle aggregation and fusion was also observed. The example prepares particles comprising 10% liraglutide by total weight of particles. The comparative example prepared particles without the hydrophilic size modulating agent. The effect of absence of this agent on the composition was studied. The results are presented in FIG. 2(a), 3(a) and in the tables below.

### Example 1

| S. No. | Ingredients |
|---|---|
| 1 | Liraglutide |
| 2 | Tromethamine |
| 3 | Protamine sulphate |
| 4 | Water for Injection |
| 4 | PLGA 50:50 |
| 5 | Dichloromethane (MDC) |
| 6 | Dimethicone |
| 7 | n-Heptane |
| 8 | Di-ethyl ether |
| 9 | n-Heptane for In-oil drying/solvent extraction |
| 10 | N₂ Drying at 2-8° C. for 0.5-1 h |
| 11 | 1% w/v Tween 80 solution in water |
| 12 | N₂ Drying at 25 ± 3° C. ° C. for 15-18 h |

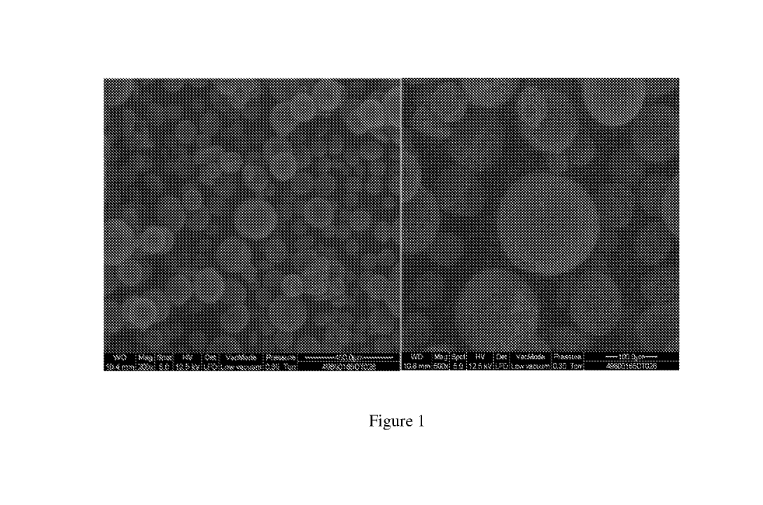

Liraglutide (100 mg), tromethamine (10 mg) and protamine sulfate (1 mg) were dissolved in water for injection (q.s. to 1 gm) to form the inner aqueous phase. The oil phase was prepared by dissolving PLGA (889 mg) in 13.93 g MDC. The aqueous phase was added slowly to prepare primary emulsion in the oil phase. To this primary emulsion, 20.89 g Dimethicone was added immediately after primary emulsification under stirring to form the secondary emulsion (W/O/O). A solvent mixture was prepared by adding n-Heptane 10.95% w/v in di-ethyl Ether (ratio 1:9). To this mixture, the secondary emulsion was added under stirring. 40 g of n-Heptane (2-8° C.) was added to above mixture. n-Heptane and di-ethyl Ether mixture was then decanted. Fresh n-Heptane (100 g) was added at an ambient temperature over the sediment particles and stirred followed by decantation. This process was repeated twice. Finally, particles suspension in n-heptane were passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. The suspension was passed through 5 micron screen. Particles collected on the screen were dried and collected into a collection vessel. The example prepares particles comprising 10% liraglutide by total weight of particles. Scanning electron microscopy (SEM) images were taken by spreading a small quantity of particles over the stub and observing under the FEI Quanta 200 electron microscope. Particles of Example 1 shows non-porous, squeezed together with few smooth particle morphology (see FIG. 1 for particles at different resolution images

### Example 2

| S. No. | Ingredients |
|---|---|
| 1 | Liraglutide |
| 2 | Tromethamine |
| 3 | Poloxamer 188 |
| 4 | Water for Injection |
| 4 | PLGA 50:50 |
| 5 | Dichloromethane (MDC) |
| 6 | Dimethicone |
| 7 | n-Heptane |
| 8 | Di-ethyl ether |
| 9 | n-Heptane for In-oil drying/solvent extraction |
| 10 | N₂ Drying at 2-8° C. for 0.5-1 h |
| 11 | 1% w/v Tween 80 solution in water |
| 12 | N₂ Drying at 25 ± 3° C. ° C. for 15-18 h |

Liraglutide (100 mg), tromethamine (10 mg) and poloxamer (20 mg) were dissolved in water for injection (q.s. to 1 gm) to form the inner aqueous phase. The oil phase was prepared by dissolving PLGA (870 mg) in 13.63 g MDC. The aqueous phase was added slowly to prepare primary emulsion in the oil To this primary emulsion, 20.45 g Dimethicone was added immediately after primary emulsification under stirring to form the secondary emulsion (W/O/O). A solvent mixture was prepared by adding n-Heptane 10.95% w/v in di-ethyl Ether (ratio 1:9) To this mixture, the secondary emulsion was added under stirring 40 g of n-Heptane (2-8° C.) was added to above mixture. n-Heptane and di-ethyl Ether mixture was then decanted. Fresh n-Heptane (98.28 g) was added at an ambient temperature over the sediment particles and stirred followed by decantation. This process was repeated twice. Finally, particles suspension in n-heptane were passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. The suspension was passed through 5 micron screen. Particles collected on the screen were dried under a constant purge of nitrogen gas and collected into a collection vessel. The example prepares particles comprising 10% liraglutide by total weight of particles.

### Example 3

| S. No. | Ingredients |
|---|---|
| 1 | Liraglutide |
| 2 | Tromethamine |
| 3 | Poly L-Lysine |
| 4 | Water for Injection |
| 5 | PLGA 50:50 |
| 6 | Dichloromethane (MDC) |
| 7 | Dimethicone |
| 8 | n-Heptane |
| 9 | Di-ethyl ether |
| 10 | n-Heptane for In-oil drying/solvent extraction |
| 11 | N₂ Drying at 2-8° C. for 0.5-1 h |
| 12 | 1% w/v Tween 80 solution in water |
| 13 | N₂ Drying at 25 ± 3° C. ° C. for 15-18 h |

Liraglutide (100 mg), tromethamine (10 mg) and Poly L-Lysine (0.93 mg) were dissolved in water for injection (q.s. to 1.04 gm) to form the inner aqueous phase. The oil phase was prepared by dissolving PLGA (890 mg) in 13.94 g MDC. The aqueous phase was added slowly to prepare primary emulsion in the oil phase. To this primary emulsion, 20.92 g Dimethicone was added immediately after primary emulsification under stirring to form the secondary emulsion (W/O/O). A solvent mixture was prepared by adding n-Heptane 10.95% w/v in di-ethyl Ether (ratio 1:9). To this mixture, the secondary emulsion was added under stirring using an overhead stirrer. The stirring was continued for 1 h. 40 g of n-Heptane (2-8° C.) was added to above mixture. n-Heptane and di-ethyl Ether mixture was then decanted. Fresh n-Heptane (100.5 g) was added at an ambient temperature over the sediment particles and stirred followed by decantation. This process was repeated twice. Finally, particles suspension in n-heptane were passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. The suspension was passed through 5 micron screen. Particles collected on the screen were dried under a constant purge of nitrogen gas and collected into a collection vessel. The example prepares particles comprising 10% liraglutide by total weight of particles.

### Example 4

| S. No. | Ingredients |
|---|---|
| 1 | Liraglutide |
| 2 | Tromethamine |
| 3 | Poloxamer 188 |
| 4 | Water for Injection |
| 5 | PLGA 50:50 |
| 6 | Dichloromethane (MDC) |
| 7 | Dimethicone |
| 8 | n-Heptane |
| 9 | Di-ethyl ether |
| 10 | n-Heptane for In-oil drying/solvent extraction |
| 11 | N2 Drying at 2-8° C. for 0.5-1 h |
| 12 | 1% w/v Tween 80 solution |
| 13 | N2 Drying at 25 ± 3° C. ° C. for 15-18 h |

Liraglutide (150 mg), tromethamine (21.6 mg) and poloxamer 188 (20 mg) were dissolved in water for injection (q.s. to 1.4 gm) to form the inner aqueous phase. The oil phase was prepared by dissolving PLGA (808 mg) in 17.15 g MDC. The aqueous phase was added slowly to prepare primary emulsion in the oil phase. To this primary emulsion, 25.73 g Dimethicone was added immediately after primary emulsification under stirring to form the secondary emulsion (W/O/O). A solvent mixture was prepared by adding n-Heptane 10.91% w/v in di-ethyl Ether (ratio 1:9). To this mixture, the secondary emulsion was added under stirring. 40 g of n-Heptane (2-8° C.) was added to above mixture. n-Heptane and di-ethyl Ether mixture was then decanted.

Fresh n-Heptane (123.69 g) was added at an ambient temperature over the sediment particles and stirred followed by decantation. This process was repeated twice. Finally, particles suspension in n-heptane were passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. The suspension was passed through 5 micron screen. Particles collected on the screen were dried and collected into a collection vessel. The example prepares particles comprising 15% liraglutide by total weight of particles.

### Example 5

| S. No. | Ingredients |
|---|---|
| 1 | Liraglutide |
| 2 | Tromethamine |
| 3 | Poloxamer 188 |
| 4 | Water for Injection |
| 5 | PLGA 50:50 |
| 6 | Dichloromethane (MDC) |
| 7 | Dimethicone |
| 8 | n-Heptane |
| 9 | Di-ethyl ether |
| 10 | n-Heptane for In-oil drying/solvent extraction |
| 11 | N2 Drying at 2-8° C. for 0.5-1 h |
| 12 | 1% w/v Tween 80 solution |
| 13 | N2 Drying at 25 ± 3° C. ° C. for 15-18 h |

Liraglutide (200 mg), tromethamine (27.3 mg) and poloxamer 188 (20 mg) were dissolved in water for injection (q.s. to 1.4 gm) to form the inner aqueous phase. The oil phase was prepared by dissolving PLGA (753 mg) in 18.06 g MDC. The aqueous phase was added slowly to prepare primary emulsion in the oil phase. To this primary emulsion, 27.1 g Dimethicone was added immediately after primary emulsification under stirring to form the secondary emulsion (W/O/O). A solvent mixture was prepared by adding n-Heptane 10.94% w/v in di-ethyl Ether (ratio 1:9). To this mixture, the secondary emulsion was added under stirring. 40 g of n-Heptane (2-8° C.) was added to above mixture. n-Heptane and di-ethyl Ether mixture was then decanted.

Fresh n-Heptane (130.25 g) was added at an ambient temperature over the sediment particles and stirred followed by decantation. This process was repeated twice. Finally, particles suspension in n-heptane were passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. The suspension was passed through 5 micron screen. Particles collected on the screen were dried and collected into a collection vessel. The example prepares particles comprising 20% liraglutide by total weight of particles.

### Example 6

### Preparation of Lyophilized Mixture Containing Liraglutide, Arginine and Poloxamer

| S. No. | Ingredients | Quantity % w/w |
|---|---|---|
| 1 | Liraglutide (acetate) equivalent to liraglutide | 2.5 |
| 2 | Arginine | 0.53 |
| 3 | Poloxamer 188 | 0.25 |
| 3 | Water for Injection | Qs to 100% |

One half of the required amount of arginine was dissolved in water for injection under gentle stirring. Poloxamer 188 was added to this solution and dissolved with stirring. Liraglutide acetate was added to above solution with stirring. Remaining amount of arginine was added while stirring until a pH of 7.0-7.5 was obtained and the solution was clear. The aqueous solution was then filtered with 0.2 μm membrane filter, filled into vials and lyophilized. The assay for liraglutide in the lyophilized mixture was 101.0% when analysed by HPLC.

### Example 7

| S. No. | Ingredients |
|---|---|
| 1 | Lyophilized mixture of Example 6 |
| 2 | Water for Injection |
| 3 | PLGA 50:50 |
| 4 | Dichloromethane (MDC) |
| 5 | Dimethicone |
| 6 | n-Heptane |
| 7 | Di-ethyl ether |
| 8 | n-Heptane for In-oil drying/solvent extraction |
| 9 | N2 Drying at 2-8° C. for 0.5-1 h |
| 10 | 1% w/v Tween 80 solution |
| 11 | N2 Drying at 25 ± 3° C. ° C. for 15-18 h |

Lyophilized mixture obtained in example 6 (262.3 mg) was dissolved in water for injection (q.s to 3 g) to form the inner aqueous phase. The oil phase was prepared by dissolving PLGA (737 mg) in MDC (17.07 g). The aqueous phase was added slowly to prepare primary emulsion in the oil phase. To this primary emulsion Dimethicone (26.56 g) was added immediately after primary emulsification under stirring using a magnetic stirrer to form the secondary emulsion (W/O/O). A solvent mixture was prepared by adding n-Heptane (10.96% w/v) in di-ethyl Ether (ratio 1:9). To this mixture, the secondary emulsion was added under stirring. 40 g of n-Heptane (2-8° C.) was added to above mixture. n-Heptane and di-ethyl Ether mixture was then decanted. Fresh n-Heptane (127.66 g) was added at an ambient temperature over the sediment particles and stirred followed by decantation. This process was repeated twice. Finally, particles suspension in n-heptane were passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. The suspension was passed through 5 micron screen. Particles collected on the screen were dried and collected into a collection vessel. The example prepares particles comprising 20% liraglutide by total weight of particles. With the use of lyophilized mixture of example 8, it was possible to make particles with higher concentration of liraglutide as exemplified herein.

### Example 8

PLGA purification method: PLGA 50:50 DL4A (10 g) was dissolved in 20 mL acetone with stirring 15-30 min Water for injection (WFI, 60 ml) was added slowly to the above solution under stirring and stirring continued for 30 min. Resulting PLGA mass was allowed to settle over 30 min and the solvent decanted. The above process was repeated twice. Following this, the same process was repeated with 60 mL of Dichloromethane (MDC), instead of acetone. MDC layer was then separated from the aqueous layer and filtered through 0.45μ filter. The MDC was distilled off using Rota-vapor at 50° C. under mild vacuum (500-600 mbar) followed by degassing the contents under high vacuum (2-20 mbar) for seven hours. After this, heating was stopped and degassing was continued for 10 hours at room temperature at 2-20 mbar vacuum. Finally, dry polymer was collected. Residual monomer was evaluated using the gas chromatography before and after the purification of PLGA. As can be seen from the table below, the process led to reduction in D,L-lactide and glycolide impurities to less than 0.1% of the weight of PLGA.

| | Residual monomer (% w/w) | |
|---|---|---|
| PLGA grade | D,L-Lactide | Glycolide |
| PLGA 50:50 DL4A-before purification | 1.04 | 0.16 |
| PLGA 50:50 DL4A - After Purification | 0.02 | ND |

*ND: not detected

### Example 9

### Preparation of Particles Using PLGA after Purification to Reduce the Levels of D,L-Lactide and Glycolide Monomers

| S. No. | Ingredients |
|---|---|
| 1 | Liraglutide |
| 2 | Tromethamine |
| 3 | Poloxamer 188 |
| 4 | Water for Injection |
| 5 | PLGA 50:50 - After purification |
| 6 | Dichloromethane (MDC) |
| 7 | Dimethicone |
| 8 | n-Heptane |
| 9 | Di-ethyl ether |
| 10 | n-Heptane for In-oil drying/solvent extraction |
| 11 | N2 Drying at 2-8° C. for 0.5-1 h |
| 12 | 1% w/v Tween 80 solution |
| 13 | N2 Drying at 25 ± 3° C. ° C. for 15-18 h |

Liraglutide (300 mg), tromethamine (30 mg) and poloxamer 188 (60 mg) were dissolved in water for injection (q.s. to 3 gm) to form the inner aqueous phase. The oil phase was prepared by dissolving PLGA (2.61 g) in 40.89 g MDC. The aqueous phase was added slowly to prepare primary emulsion in the oil phase. To this primary emulsion, 61.34 g Dimethicone was added immediately after primary emulsification under stirring to form the secondary emulsion (W/O/O). A solvent mixture was prepared by adding n-Heptane 10.95% w/v in di-ethyl Ether (ratio 1:9). To this mixture, the secondary emulsion was added under stirring. 120 g of n-Heptane (2-8° C.) was added to above mixture. n-Heptane and di-ethyl Ether mixture was then decanted. Fresh n-Heptane (294.83 g) was added at an ambient temperature over the sediment particles and stirred followed by decantation. This process was repeated twice. Finally, particles suspension in n-heptane were passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. The suspension was passed through 5 micron screen. Particles collected on the screen were dried and collected into a collection vessel. The example prepares particles comprising 10% liraglutide by total weight of particles. The particles were prepared according to this example using a purified PLGA of example 8, containing less than 0.1% of D,L-lactide and glycolide monomer impurity. The particles were analysed by HPLC as provided in example 10 below.

### Example 10

Assay of liraglutide and related substances from particles of Example 2 and 9 was determined using reversed phase high performance liquid chromatography using C18 as stationary phase. The samples were prepared using a mixture of phosphate buffer pH 7.8 and organic mixture of DMSO and methanol (6:2:2). Sample concentration was kept 600 μg/ml. Separation was carried out on stationary phase Poroshell EC-18 (150×4.6 mm, 2.7μ) by using 0.025 molar potassium phosphate buffer pH 1.5. and acetonitrile. Buffer and Acetonitrile (9:1) and Buffer and acetonitrile (3:7) as mobile phase A and B respectively were used. The detection wavelength was 210 nm and elution at 0.8 mL/min (Gradient). The column was thermostated at 40° c. and auto-sampler temperature was kept at 5° C. Quantitation was done using external standard by injecting 0.1% solution of liraglutide. RRT of Impurity A and B were 0.88 and 1.19 respectively. The RRF of Impurity A and B were 1.18 and 1.19 respectively. 20 μl of clear test and standard solutions were injected in above chromatographic system. Retention time for liraglutide peak was about 59 minutes and total run time was 125 minutes.

| | | | Related Substances | | | |
|---|---|---|---|---|---|---|
| | | | | | Highest | Total (Known + |
| Sr. | Example | Assay | Impurity A | Impurity B | Unknown | Unknown) |
| No. | no. | (%) | (%) | (%) | (%) | (%) |
| 1 | Example 2 | 96.22 | ND | 3.786 | 1.652 | 7.598 |
| 2 | Example 9 | 97.82 | ND | 0.19 | 0.41 | 1.28 |

ND: not detected

When the particles prepared using PLGA before purification (example 2) were compared with the particles prepared using purified PLGA (example 9), for the content of liraglutide, it was observed that for particles of example 9, total impurities as well as the each of known and unknown impurities of liraglutide were reduced to substantial levels. (see table above). When the content of residual monomer was less than 0.1%, the total impurity levels were below 3% and Individual impurities were less than 0.5% 9, as calculated from the HPLC peaks,

### Example 11

| S. No. | Ingredients |
|---|---|
| 1 | Liraglutide |
| 2 | Tromethamine |
| 3 | Protamine sulfate |
| 4 | Water for Injection |
| 5 | PLGA 50:50 |
| 6 | Dichloromethane (MDC) |
| 7 | Dimethicone |
| 8 | n-Heptane |
| 9 | Di-ethyl ether |
| 10 | n-Heptane for In-oil drying/solvent extraction |
| 11 | N2 Drying at 2-8° C. for 0.5-1 h |
| 12 | 1% w/v Tween 80 solution |
| 13 | N2 Drying at 25 ± 3° C. ° C. for 15-18 h |

Liraglutide (400 mg), tromethamine (40 mg) and protamine sulfate (4 mg) were dissolved in water for injection (q.s. to 2.3 gm) to form the inner aqueous phase. The oil phase was prepared by dissolving PLGA (1.55 g) in 24.37 g MDC. The aqueous phase was added slowly to prepare primary emulsion in the oil phase. To this primary emulsion, 36.57 g Dimethicone was added immediately after primary emulsification under stirring to form the secondary emulsion (W/O/O). A solvent mixture was prepared by adding n-Heptane 10.95% w/v in di-ethyl Ether (ratio 1:9). To this mixture, the secondary emulsion was added under stirring. 80 g of n-Heptane (2-8° C.) was added to above mixture. n-Heptane and di-ethyl Ether mixture was then decanted. Fresh n-Heptane (175.8 g) was added at an ambient temperature over the sediment particles and stirred followed by decantation. This process was repeated twice. Finally, particles suspension in n-heptane were passed through 5 micron screen. Particles collected on the screen were dried under a constant purge of nitrogen gas and collected into a collection vessel. The collected particles were dispersed in 1% w/v Tween-80 solution (1 g in 10 ml) followed by water for injection over a 5 micron screen. The separated particles were suspended in 1 g/10 ml Water for injection. The suspension was passed through 5 micron screen. Particles collected on the screen were dried and collected into a collection vessel. The example prepares particles comprising 20% liraglutide by total weight of particles.

### Example 12

### Method for Removing the Residual Solvent and Analysis Thereof

Collected particles of Example 11 were treated with aqueous solution containing 10% w/v propylene glycol and 0.05% w/v Tween 80 (1 g in 25-50 ml) with stirring at 500-1000 rpm at 35±3° C. for 2-4 hr. The particles were collected on 5 micron screen, followed by rinsing with water for injection. Finally, particles were dried and transferred into a collection vessel.

The concentration of residual solvents were evaluated using the gas chromatography and the concentration levels were classified based on the ICH guideline Q3C Impurities: Guideline for Residual Solvents, by its toxicity grade. According to this guideline, the dichloromethane is codified as class 2 solvent with a permitted daily exposure (PDEs) limit of no more than 6 mg/day (concentration limit: not more than 600 ppm), while the Di-ethyl ether and n-Heptane is codified as Class 3 (low toxicity) solvents with a PDEs of no more than 50 mg/day (concentration limit: not more than 5000 ppm). As observed from table below, the residual solvent levels in the particles of the present invention, after treatment to remove the said residual solvents, was within the acceptable levels of ICH guidelines.

| | Residual solvents level | | |
|---|---|---|---|
| | Dichloromethane | Di-ethyl ether | n-Heptane |
| Example no. | (NMT 600 ppm) | (NMT 5000 ppm) | (NMT 5000 ppm) |
| Example 11 | ND | 4828 | 4140 |

### Example 13

Particle sizes of different particles prepared in examples above using different hydrophilic size modulating agent were evaluated as in-process analytical tool by the Nikon optical microscopy (see FIGS. 2(a), (b), (c) and (d)).

| | | | Hydrophilic particle | Mean |
|---|---|---|---|---|
| | | % w/w | size modulating | particle |
| Sr. No. | Example no. | drug loading | agent | size (μm) |
| 1. | Comparative | 10 | None | >100 |
| | Example 1 | | | |
| 2. | Example 1 | 10 | Protamine sulfate | 60.45 |
| 3. | Example 2 | 10 | Poloxamer | 55.29 |
| 4. | Example 3 | 10 | Poly L-Lysine | 49.18 |
| 5. | Example 4 | 15 | Poloxamer | 67.37 |
| 6. | Example 5 | 20 | Poloxamer | 98.51 |

As demonstrated by FIG. 2(a) the comparative example with no hydrophilic particle size modulating agent demonstrates larger particles as compared to examples 1, 2 and 3 as demonstrated in FIGS. 2(b), 29c) and 2(d) respectively. Furthermore, as demonstrated in FIG. 3(a), the particles prepared according to comparative example 1 stuck to the walls of the container, which did not happen with the particles of example 1 as seen in FIG. 3(b).

### Example 14

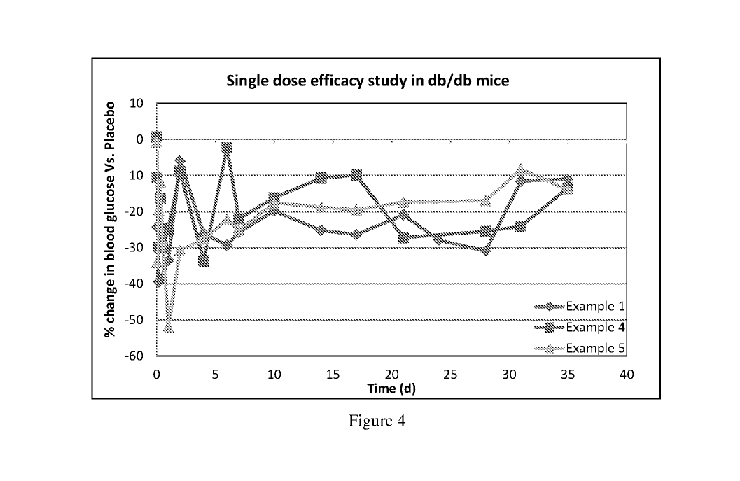

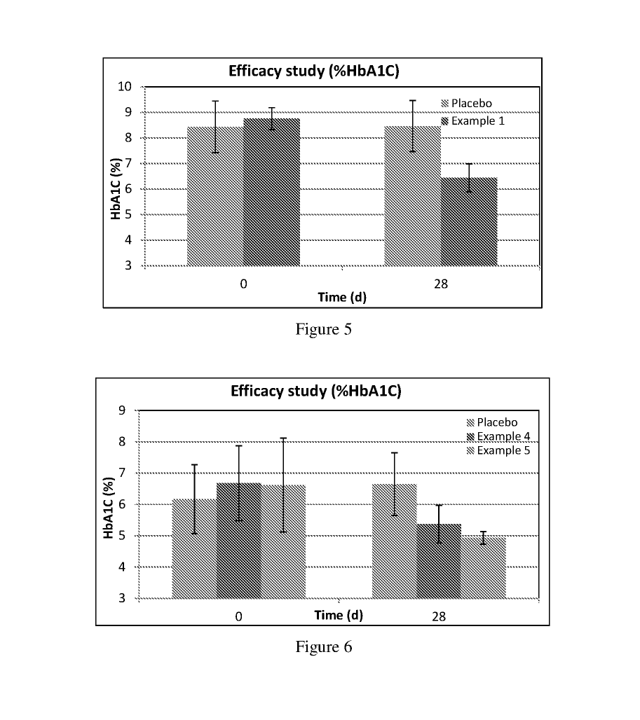

Preclinical efficacy study was performed on the db/db mice. All the animals were acclimatized for 5 day. On day 0, each animal was weighed and approximately 10 μL of blood was collected from retro-orbital plexus and blood glucose concentration was measured with glucose strips using Blood Glucose Meter (Blood Glucose Meter, One Touch™ Ultra™; LIFESCAN, Johnson & Johnson) which was considered as baseline value (0 hour). All the animals were randomized into treatment groups containing 4 male and 4 female animals each on the basis of baseline. The dose volume for each animal was calculated based on their respective body weights. Single dose of composition of Example 1, 4 and 5 was injected at 20 mg/kg liraglutide. The blood was collected at different intervals. The blood glucose levels was analysed using PRISM (Graph Pad version 5.04) by Student's paired t test as compared to 0 hour blood glucose value. HbA1c was measured at initial and end of the study (28 d) using (Hemoglobin AC1 kits (Bio System, Spain). Liraglutide composition of Example 1, 4 and 5 showed significant reduction in mean blood glucose (mg/dL) levels up to 1 month at 20 mg/kg compared to placebo (See FIG. 4). The Mean % HbA1C at 28 day of treatment for Example 1 was 6.44% and was found to be significantly decreased compared to initial (8.75%) and placebo (8.46%) at 28 day of treatment (FIG. 5). The HbA1C results are shown in FIG. 6 for compositions of examples 4 and 5. Mean % HbA1C reduction of Example 4 & 5 was 1.28% and 1.69%, respectively, as compared to placebo, which is significant reduction in % HbA1C after 1-month of injection.

1. A composition comprising particles, wherein said particles comprise
a) Poly(lactide-co-glycolide) polymer;
b) therapeutically effective amount of Liraglutide or a pharmaceutically acceptable salt thereof; and
c) a hydrophilic particle size modulating agent,
wherein, the composition is free of added divalent metal ions.

2. The composition of claim 1, wherein the hydrophilic particle size modulating agent is protamine or its pharmaceutically acceptable salt.

3. The composition of claim 2, wherein protamine or its pharmaceutically acceptable salt is present in an amount from about 0.01% w/w to about 1% w/w.

4. The composition of claim 1, wherein the hydrophilic particle size modulating agent is poloxamer.

5. The composition of claim 4, wherein poloxamer is present in an amount from about 0.1% w/w to about 5% w/w.

6. The composition of claim 1, wherein the hydrophilic particle size modulating agent is poly L-lysine.

7. The composition of claim 6, wherein poly L-lysine is present in an amount from about 0.01% w/w to about 1% w/w.

8. The composition of claim 1, wherein liraglutide is present as a salt with a base.

9. The composition as in claim 8, wherein liraglutide is present as a tromethamine salt.

10. The composition of claim 1, wherein the mean particle size is between 5-100 μm.

11. The composition of claim 1, wherein said Poly(lactide-co-glycolide) polymer has a lactide:glycolide ratio of about 50:50.

12. The composition of claim 1, wherein D.L-lactide and glycolide monomer are each less than 0.1% w/w of Poly(lactide-co-glycolide) polymer.

13. The composition of claim 12, wherein the total impurities of liraglutide is less than 3% as determined by HPLC.

14. The composition of claim 1, wherein liraglutide is present from about 5% w/w to about 40% w/w of the total weight of the particles.

15. The composition of claim 1, wherein liraglutide is present in an amount from about 5% w/w to about 15% w/w of the total weight of the particles.

16. The composition of claim 1, wherein liraglutide is present in an amount between 16% w/w to about 25% w/w of the total weight of the particles.

17. The composition of claim 1 wherein the levels of class II residual solvents is less than 600 ppm and the level of Class III residual solvents is less than 5000 ppm.

18. A method of reducing blood glucose level for a period of at least one month in a patient in need thereof comprising administering a composition of claim 1.

19. A method of reducing blood glucose level for a period of at least one month in a patient in need thereof comprising administering a composition of claim 11.

20. A method of reducing blood glucose level for a period of at least one month in a patient in need thereof comprising administering a composition of claim 14.
